(19) United States
(12) Patent Application Publication
(10) Pub. No.: US 2015/0044665 A1
(43) Pub. Date: Feb. 12, 2015

(54) TARGET-SPECIFIC PROBE COMPRISING T7 BACTERIOPHAGE AND DETECTING FOR BIOMARKER USING THE SAME

(71) Applicant: KOREA INSTITUTE OF SCIENCE AND TECHNOLOGY, Seoul (KR)

(72) Inventors: Kwan-Hyi LEE, Goyang (KR); Jong-Hoon CHOI, Seoul (KR); Jong-Wook LEE, Anyang (KR); Hyun-Kwang SEOK, Seoul (KR); Mintai Peter HWANG, Seoul (KR); Jang-won SONG, Seoul (KR)

(73) Assignee: KOREA INSTITUTE OF SCIENCE AND TECHNOLOGY, Seoul (KR)

(21) Appl. No.: 14/061,011

(22) Filed: Oct. 23, 2013

(30) Foreign Application Priority Data
Aug. 9, 2013 (KR) 10-2013-0094865

(51) Int. Cl. G01N 33/554; G01N 33/58; C12N 15/10; C12N 7/00

(52) U.S. Cl. CPC G01N 33/554; C12N 7/00; G01N 33/588; C12N 15/1037; G01N 2333/01; C12N 2795/10221; C12N 2795/10231
USPC 435/5; 435/235.1

(57) ABSTRACT

The present invention relates to a target-specific probe containing T7 bacteriophage with a targeting antibody, and a detection method or a detection kit for a biomarker using the target-specific probe. The biomarker can be detected by using the genetically-modified T7 bacteriophage expressing various heterogeneous proteins and peptides on its surface and antibody-antigen specific reaction which can make the probe targeted to a biomarker or bacteria; and a detectable labeling agent, for example a quantum dot.

### CROSS-REFERENCE TO RELATED APPLICATIONS

This application claims the benefit of Korean Patent Application No. 10-2013-0094865, filed on Aug. 9, 2013, in the Korean Intellectual Property Office, the entire disclosure of which is incorporated herein by reference.

### FIELD OF THE INVENTION

The present invention relates to a target-specific probe including T7 bacteriophage and a targeting antibody, and a detection method, a quantification method and a detection kit of a biomarker using the probe.

### DESCRIPTION OF THE RELATED ART

A biomarker is a kind of biomaterial being present in biological or medical specimens, which functions as a marker being capable of diagnosing the condition of a disease by detecting a change in the structure or the concentration thereof qualitatively and/or quantitatively and determining the treatment effects of a medicine and the correlation with other diseases comprehensively. For the early diagnosis of diseases, it is essential to analyze a biomarker being presented at the beginning stage of the diseases quantitatively. However, the technologies being currently available for monitoring the diseases do not properly meet the technical needs for the early diagnosis of diseases because of the limits such as sensitivity, quarantine speed, and costs.

ELISA (Enzyme-Linked ImmunoSorbent Assay), western blotting, and a mass spectrometry-based method have been generally used to quantitatively analyze biomarkers. ELISA has a difficulty in accurate detection, because of the reaction blocking by polysaccharides or phenol compounds in the test samples or the low concentration of bacteriophages in tissues. While the mass spectrometry-based method has a very good sensitivity so that it is applicable to analyze a small amount of a biomarker, it has difficulty in securing reproducibility due to use of chromatography method and also has a huge deviation of analysis data due to machine errors. In addition, this method requires excessive labor and long time.

Recently, as nanotechnologies are developed rapidly, there is an emerging detection technology which can be applied to the sample undetected by previous detection method. For example, Lieber et. al from Harvard University published a nano-sensor for detecting a single bacteriophage (Science, vol. 329, pp. 830-4, Aug. 13 2010), and Mirkin et. al from Northwestern University established Nanosphere company, published a molecule detection technology using a nanoprobe (Sensors, vol. 12, pp. 1657-1687, Feb. 7 2012).

However, the method using the nanoparticle has good sensitivity but a difficulty in approaching a sample of interest for quantitatively measuring a very small amount (see FIG. 1) and detecting a relatively larger sample such as virus or bacterial cell than the nanoparticle. Thus, the application of method is very restricted to the detection of a protein and blood glucose (FIG. 2).

A quantum dot is an inorganic semiconductive substance having a nano-size, which has been recently applied to various medical engineering fields, because of its excellent optical properties including high quantum efficiency, excellent resistance to photo fading, the control of fluorescence property by size, and non-overlapped fluorescence spectrum. Attempts for using quantum dots have been made for the quantification of important disease markers (Analytical Chemistry, vol. 76, pp. 4806-4810, Aug. 15 2004; Analytical Chemistry, vol. 82, pp. 5591-5597, Jul. 1 2010).

Furthermore, in order to overcome the quenching phenomenon where the fluorescence intensity of quantum dots becomes dramatically weak, attempts to measure the number of quantum dots in a different manner were published. For example, the change of electrical conductivity due to cadmium ions which constitute quantum dots was measured by dissolving the quantum dots in a strong acid, when Prostate-specific Antigen (PSA) was separated, detected, and quantified (Small, vol. 4, pp. 82-86, January 2008). However, there was an issue that a large amount of toxic cadmium ions were generated. In another analysis example using quantum dots, for the purpose of measuring intrinsic fluorescence by the separation of quantum dots, organic solvents and alkali solutions having a high concentration were used to cleave Streptavidin-Biotin bond (Analyst, vol. 135, pp. 381-389, 2010.). However, there were issues that the aggregation of quantum dots might occur due to the organic solvents and the need of various buffering solutions, because of the deterioration of the stability and optical properties of the quantum dots under experiment conditions. The method had poor reproducibility.

Therefore, in order to detect biomarkers with high sensitivity, there is a need for a new detection system of biomarker that is capable of overcoming the limits of the previous detection systems including nanoparticles and nano-elements, and that provides a simple and easily analysis, an accurate detection results and high sensitivity.

### SUMMARY OF THE INVENTION

An embodiment of the present invention is to provide a target-specific probe including a complex of T7 bacteriophage and a binding peptide bound to the T7 bacteriophage, and a targeting antibody connected to the complex, and a detection method, a quantitative analyzing method and a detection kit of a biomarker using the probe.

It is another object of the invention to provide a detection method, a quantitative analyzing method and a detection kit of a biomarker using the target-specific probe, being capable of overcoming the limits of the previous detection systems including nano-particles and nano-elements, being simply analyzed, being easily handled, and providing accurate detection results with high sensitivity.

In order to achieve the above-mentioned objects, an embodiment of the present invention is to provide a target-specific probe including a binding peptide connected to a head part of T7 bacteriophage and a targeting antibody connected to the binding peptide, and a detectable labeling agent connected to a tail part of T7 bacteriophage.

Another embodiment of the present invention including contacting the target-specific probe including a targeting antibody specific to a target biomarker of interest and a detectable labeling agent, to a sample containing the target biomarker, and detecting the detectable labeling agent of the target-specific probe targeted to the target biomarker.

In the method for detecting a biomarker, when the labeling agent is detected, the biomarker can be determined to be present in the sample, or the amount of the biomarker in the sample can be measured by preparing a standard curve of the labeling agent at various concentrations and comparing the detected amount of the biomarker-targeted labeling agent with the standard curve.

Further embodiment of the present invention relates to a detection kit including a target-specific probe and a detector for detecting a labeling agent of the targeted probe.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic diagram in which two-dimensional detection methods using a nano-element used in the prior art that shows the limit in detecting a small amount with excellent sensitivity, due to the limited accessibility to the subject to be detected.

FIG. 2 is a schematic diagram showing the disadvantages of the method using only nanoparticle in detection of a virus and bacteria due to the size limitation and the narrow application scope for a protein and blood glucose.

FIG. 3 shows a structure of T7 bacteriophage.

FIGS. 4a to 4f show PCR protocol of T7 bacteriophage gene according to an embodiment of the present invention.

FIG. 5 shows an amino acid sequence of 6× His inserted into an internal region of the tail part of T7

FIG. 6 shows a cleavage map of a vector including a modified T7 bacteriophage according to Example 2.

FIG. 7 is a schematic drawing of the genetically-modified T7 bacteriophage according to an embodiment of the present invention.

FIG. 8 and FIG. 9 are fluorescence images of cells targeted by the genetically-modified T7 bacteriophage according to an embodiment of the present invention.

FIG. 10 is a TEM photograph of the genetically-modified T7 bacteriophage according to an embodiment of the present invention.

FIG. 11 is a quantum dot concentration-fluorescence intensity related standard curve at 350 nm according to an embodiment of the present invention.

FIG. 12 is a graph showing a quantitative analysis result of CRP biomarker according to an embodiment of the present invention.

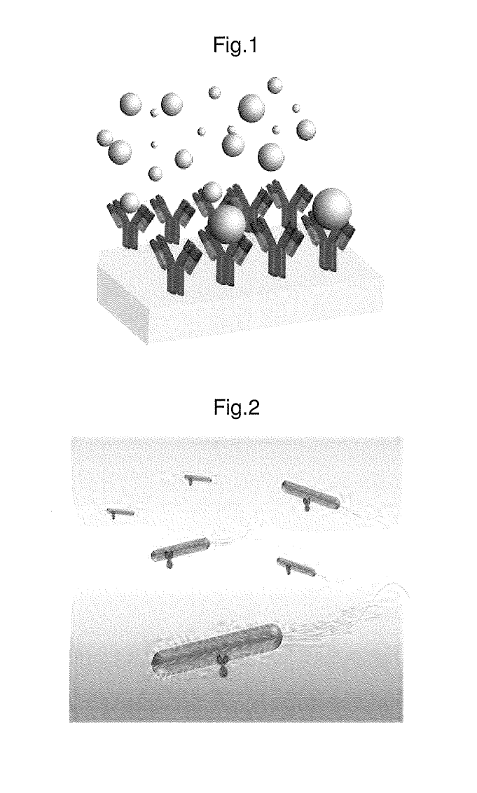

### DESCRIPTION OF THE PREFERRED EMBODIMENTS

The target-specific probe of the present invention includes a binding peptide connected to a head part of T7 bacteriophage and a targeting antibody linked to the binding peptide; and a detectable labeling agent connected to a tail part of the T7 bacteriophage.

The binding peptide aids the T7 bacteriophage in targeting to a biomarker by coupling with the targeting antibody. The binding peptide may be provided by being prepared as a separate peptide and being connected to the head part protein of T7 bacteriophage, or provided as a form of fusion protein connected to C-terminal of the head part in T7 bacteriophage. The binding peptide may be directly connected to the head part of T7 bacteriophage, or coupled using a linker peptide consisting of 15 to 30 amino acids, preferably, 15 to 25 amino acids.

Herein, the term of “Polypeptide” means a polymer chain of amino acids. The terms of “peptide” and “protein” may be used interchangeably with the polypeptide, and mean a polymer chain of amino acids. The polypeptide includes a natural or synthetic protein, a protein fragment and a polypeptide analogue of amino acid sequence. The polypeptide may be a single polymer or complex with other polymer.

The examples of binding peptide include protein G(Pro G), protein A(ProA), protein A/G, Fc receptor, protein Z (Pro Z) and a biotinylation tag, but not limited thereto. For more specific examples, the binding peptide may be protein G including an amino acid sequence of 195 amino acids as represented by SEQ ID NO: 1, or protein A including an amino acid sequence of 508 amino acids as represented by SEQ ID NO: 2.

The targeting antibody or the labeling agent may be connected to the head part or the tail part of T7 bacteriophage using a linker selected from HIS tag, CYS tag, GST tag, and biotinylation tag. The biotinylation tag includes avidin, streptavidin or a biotin-binding peptide including an amino acid sequence of SEQ ID NO: 3 (LAAIPGAGLIGTH), but not limited thereto. The tag may be provided by a fusion protein of the tag which is inserted into an internal region of the head part or the tail part of T7 bacteriophage or connected to a terminus of the head part or the tail part in T7 bacteriophage.

Preferably, the targeting antibody may be connected to the binding peptide coupled with the head part of T7 bacteriophage, and the labeling agent may be connected to the tail part of T7 bacteriophage via a tag peptide connected to the tail part of T7 bacteriophage.

In the target-specific probe of the present invention, T7 bacteriophage is used as a sensor platform which couples with the targeting antibody and the labeling agent. For example, when wild type T7 bacteriophage is used, the target-specific probe may be produced by coupling the binding protein with the head part of T7 bacteriophage; coupling the targeting antibody with the binding peptide directly or with using a linker peptide; and coupling a labeling agent to the tail part of T7 bacteriophage directly or through a tag. SEQ ID NOs:4 and 5 represent the nucleotide sequence of wild type T7 bacteriophage's head part(1008 bp) and nucleotide sequence of wild type T7 bacteriophage's tail part(1662 bp), respectively. The nucleotide sequence of head part includes a nucleotide sequence of 19363 to 20370 bases in full length T7 bacteriophage, and the nucleotide sequence of tail part includes a nucleotide sequence of 30936 to 35597 bases in full length of T7 bacteriophage.

Alternatively, when the genetically-modified T7 bacteriophage is used as sensor platform, the binding protein was provided as a fusion protein connected with C-terminal of T7 bacteriophage's head part protein directly or through a linker peptide, and a tag peptide is provided as a fusion protein inserted into the tail part of T7 bacteriophage or connected to C-terminus of the tail part, and a labeling agent is connected to the tag, to produce the target specific probe. For binding the tag with the tail part of T7 bacteriophage, the gene encoding the tail and the both ends of the tag are introduced by two restriction sites, cleaved by the restriction enzyme and ligated.

The genetically-modified T7 bacteriophage may contain the head part which includes a fusion protein of the binding peptide connected with C-terminus of the head part in T7 bacteriophage directly or through a linker peptide; and the tail part which includes a fusion protein of a tag peptide inserted into the tail part or connected to C-terminus. Therefore, the present inventors developed the target-specific probe which can be used for imaging the specific part of cell or tissue and for a quantitative sensor to precisely analyze a biomarker, and an imaging kit and a detection kit, because the head part of T7 phage is provided as targeting moiety and the tail part is provided as a quantitative indicator moiety

In an embodiment of the present invention, a method of preparing a target-specific probe (imaging/quantification sensor) including the genetically-modified T7 bacteriophage as a sensor platform, comprises the steps of preparing a T7 bacteriophage sensor plat form by genetically modifying the head part and the tail part of T7 bacteriophage; and coupling a targeting antibody with the head part and coupling a detectable labeling agent with the tail part.

The binding peptide may be bound to the targeting antibody by using a specific binding property of the binding peptide to F_{c} part of targeting antibody, or by using nonspecific binding of chemical coupling method. The F_{ab} part of targeting antibody is always active in case of using a specific binding property of the binding peptide, thereby improving the targeting property. The example of the Fc part includes Fc receptor I derived from rabbit, goat, human or mouse as F_{c} part of protein G. Specifically, the Fc part is Mus musculus (Mouse) Fc receptor I derived from mouse including an amino acid sequence as represented by SEQ ID NO: 7, or Homo sapiens (Human) F_{c} receptor I including an amino acid sequence as represented by SEQ ID NO: 8.

A specific example of the chemical coupling method may be a chemical coupling method using N-hydroxysuccinimide(NHS) and ethyl(dimethyl aminopropyl) carbodiimide(EDC)(Nat Protoc 2007, 2 (5), 1152-1165). Preferably, an excessive amount of antibodies are used for binding, because the F_{ab} part activity of targeting antibody is likely to be inhibited due to the non-specific binding to the binding peptide.

The labeling agent applicable to the invention may include a quantum dot, magnetic bead nanoparticle, gold nanoparticle, fluorescent dye, fluorescent protein, nano phosphor, or silicon nanoparticle, and the labeling agent may be detected by fluorescence microscopy, SEM, TEM, CT, MRI, etc.

The labeling agent may bind directly to a linker for coupling the labeling agent, selected from HIS tag, CYS tag, GST tag, Biotin tag, Avidin tag, and Streptavidin tag, or it may be coupled to the linker after the chemical treatment to the labeling agent in order to increase a binding ability to the linker.

The method of coupling a labeling agent with the tail part of T7 bacteriophage, for example, may use Polymerase Chain Reaction (PCR).

One (1) molecule of T7 bacteriophage can couple with one molecule of labeling agent in molar ratio of 1:1, and the target-specific probe of present invention has a high detection level and a high sensitivity, and sufficient space between the labeling agents, thereby enabling more accurate quantitative analysis than the prior art.

The labeling agent in itself may be coupled through tag connected to the tail part of the T7 bacteriophage, or it may be coupled after chemical treatment of the labeling agent to increase a binding ability to the tag.

When the labeling agent is a quantum dot, it may be used in itself, or as a surface treated one. That is, the quantum dot includes a hydrophilic surface layer obtained by treating with an amphiphilic substance containing both of hydrophilic group and hydrophobic group. Particularly, it is preferable that the quantum dots show the minimized aggregation due to the hydrophilic surface and are functionalized with the treatment of nickel. For example, the hydrophilic surface may be obtained by treating with one or more substances selected from the group consisting of 1-Myristoyl-2-Hydroxy-sn-Glycero-3-Phosphocholine (MHPC), 1,2-Distearoyl-sn-Glycero-3-Phosphoethanolamine-en-Methoxypolyethyleneglycol-2000 (DPPE-PEG2000), and 1,2-Dioleoyl-sn-Glycero-3-en-{5-amino-1-carboxylpentyl}iminodiacetic acid succinyl nickel salt (Ni-NTA). The amphiphilic substances may be treated alone or in a combination of two or more.

In particular, MHPC can be densely coated onto the surface of a sphere, since it has a single acyl group chain. DPPE-PEG2000 can confer stability on the quantum dots coated with the lipids. Ni-NTA can bind to an amino acid in the tail part of T7 bacteriophage directly or in the tag, and thus helps the quantum dot to bind to the tail part of T7

According to an embodiment of the invention, MHPC, DPPE-PEG2000, and Ni-NTA may be used alone or in a mixture. For example, 50 to 95 mole % of MHPC, 4 to 35 mole % of DPPE-PEG2000, and 1 to 15 mole % of Ni-NTA are mixed with quantum dots to make an emulsion, and are sonicated to form a hydrophilic surface layer on the surface of the quantum dots. The mixture is merely mentioned as one illustration, and can be made by selecting the kind and the mixing ratio of the lipids.

According to a further embodiment of the present invention, an imaging kit for a desired part of cell or tissue using the target-specific probe, and a method or a kit of quantitative analysis of a target biomarker in the biological samples such as blood, urine, saliva and the like.

Another aspect of the invention relates to a method for detecting a biomarker including the steps of contacting the target-specific probe containing a targeting agent specific to the biomarker and a labeling agent to a sample containing the biomarker, and detecting the labeling agent of the biomarker-targeted probe.

Also, another aspect of the invention relates to a detection kit of a biomarker including a target-specific probe and a detector for detecting a labeling agent of the targeted probe. The target-specific probe is in detail in the above.

The method for detecting a biomarker may further comprise a step in which the biomarker is determined to be present in the sample if the labeling agent is detected. Furthermore, the method further including a step of performing a quantitative analysis by comparing the amount of the detected labeling agent of the probe targeted to the biomarker, with a standard curve which is drawn with detectable amounts measured at various concentrations of the labeling agent.

The detection kit of biomarker comprises a target-specific probe and a detector for detecting a targeted probe and labeling agent.

When the target-specific probe including T7 bacteriophage is contacted with a sample and targeted, the subject biomarker can be measured quantitatively with a high sensitivity without being affected by other materials, by separating selectively the labeling agent (i.e., Quantum dot) from the T7 bacteriophage and measuring the intrinsic absorbance of the labeling agent. The intrinsic absorbance of labeling agent corresponds precisely to an amount of biomarker. The selective separation of the labeling agent from T7 bacteriophage, for example, can performed by cleaving the bond between the hydrophilic group on the surface of quantum dot and the Histidine of functionalized tail part, with the addition of imidazole.

There are several advantages of the target-specific probe using T7 bacteriophage sensor platform according to the present invention.

T7 bacteriophage probe can be targeted to an environmentally-harmful factor with a large size of 60 nm, and has shape and structure advantage of functionalized head and tail parts. Also, the probe can detect a smaller amount of the factor than two-dimensional nanoparticle, because the probe detects the environmentally-harmful factors by targeting to them three-dimensionally in medium.

T7 bacteriophage probe can perform an accurate quantitative analysis, because the targeting antibody targets to a subject material at a molar ratio of 1:1, which is achieved by coupling one targeting antibody to a head part of T7 bacteriophage probe. It is possible to measure an accurate amount of biomarker, because the number of biomarker corresponds to the number of quantitative labeling agent bound to T7 bacteriophage.

The measured concentration of biomarker is proportional to the concentration of T7 bacteriophage, because the size of T7 bacteriophage probe is fixed as 60 nm. Therefore, the measurement error becomes smaller at a quantitative analysis. Usually, the biomarker has a size of nanometer, and the probe can bind selectively to the biomarker in blood containing other proteins and chemicals.

According to an embodiment of the present invention, when the quantum dot has a hydrophilic surface treated with Ni-NTA and the modified T7 bacteriophage includes 6× His tag, an accurate quantitative analysis can be achieved, because the a loss of quantitative labeling agent in the medium change is prevented due to the bond between Ni-NTA of quantum dot and the Histidine of T7 bacteriophage. The bond between Ni-NTA and Histidine is a complex binding where a central metallic ion of Ni is surrounded by Histidine through the hydrogen bond. Therefore, it is possible to prevent aggregation of T7 bacteriophage sensor due to chemical coupling, and a loss of sensor material which can be occurred in process of changing medium.

The modified T7 bacteriophage probe may be produced in large quantities easily, because the probe can be prepared by applying the mass production method using the phage display technology at low coat. Accordingly, it is possible to produce a phage presenting various functional heterologous proteins and peptides on its surface according to the genetic recombinant technology of T7 bacteriophage, a targeting technology of the probe to a biomarker or bacterial using the antigen-antibody specific reaction, and a quantitative analyzing technology using a quantum dot.

The target specific probe using T7 bacteriophage and the detection method using the same according to the present invention can detect quantitatively a trace amount compared to two-dimensional nanoparticle; measure a biomarker accurately, as the number of biomarker corresponds to the number of quantitative leveling agent labeled on T7 bacteriophage; and prevent aggregation of T7 bacteriophage sensor due to chemical coupling and loss of sensor material which may be generated in process of changing medium.

Hereafter, the invention will be described in more detail through examples and comparative examples. However, the following examples are to merely illustrate the present invention, and the scope of the invention is not limited by them in any ways.

### Example 1

### Preparation of a T7 Bacteriophage

1-1: Preparation of Each Part of T7 Bacteriophage

The DNA of T7 bacteriophage was purchased from Merck (Merck KGaA, Darmstadt, Germany). The PCR kit used for the example was purchased from Qiagen (Qiagen, Hiden, Germany), and the restriction enzymes including Sac II were purchased from New England Biolabs (New England Biolabs, Ipswich, Mass., USA). A DNA primer and oligonucleotide were made by Cosmogenetech (Cosmogenetech, Seoul, Korea).

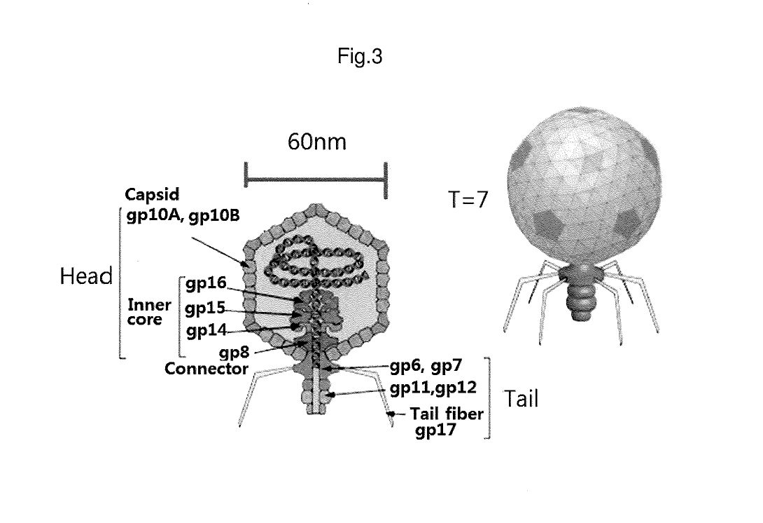

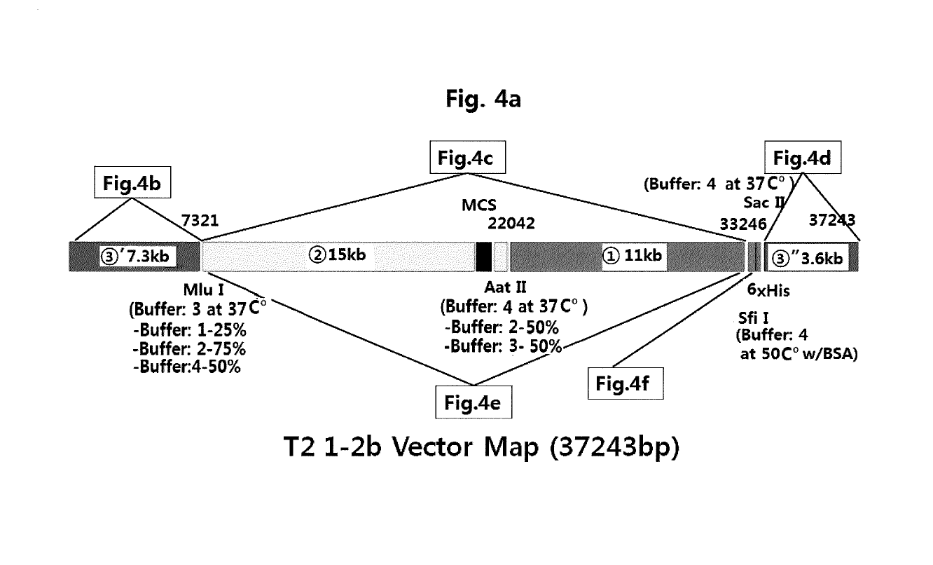

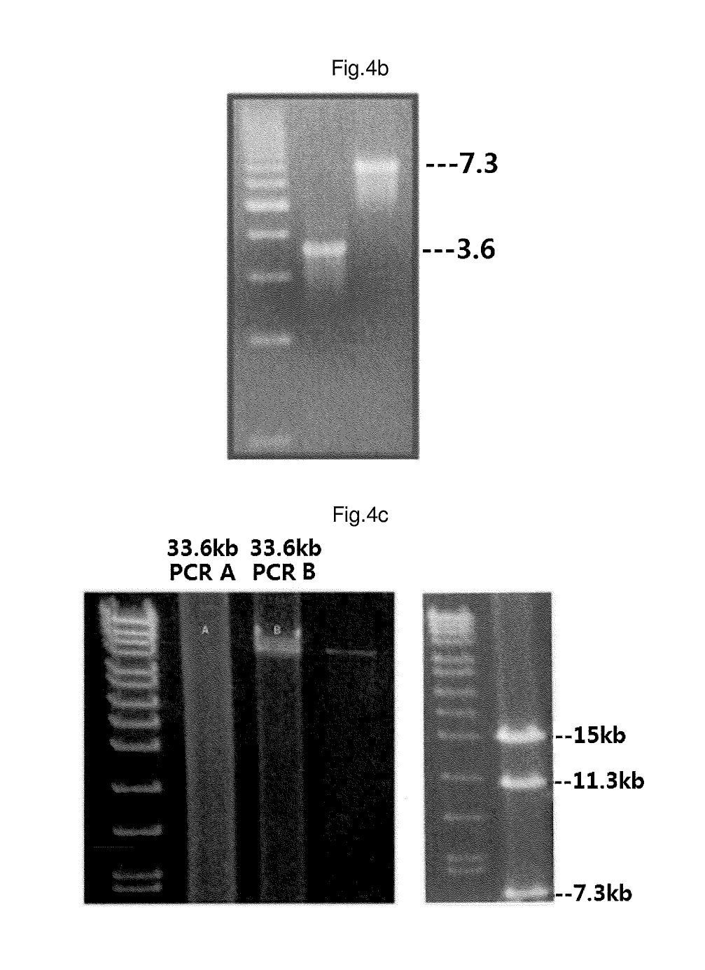

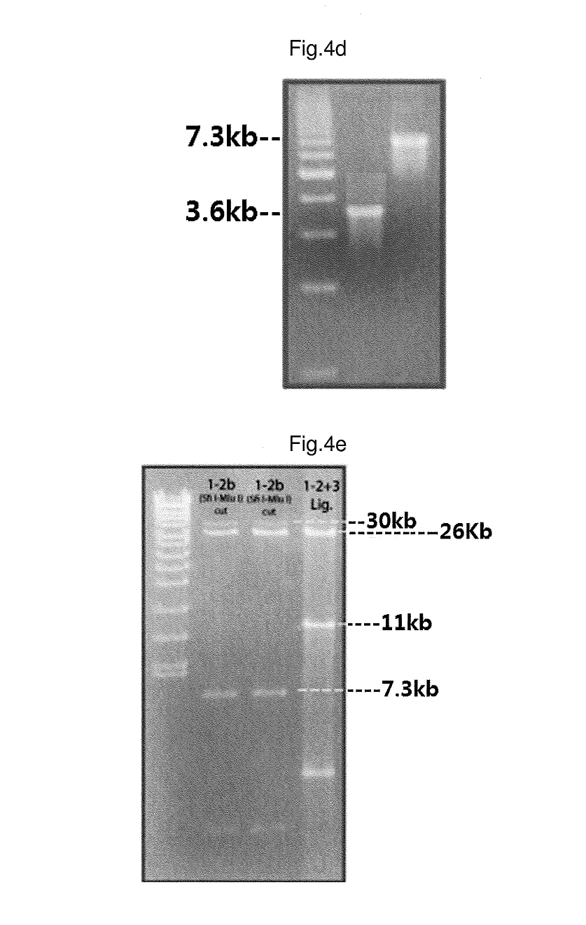

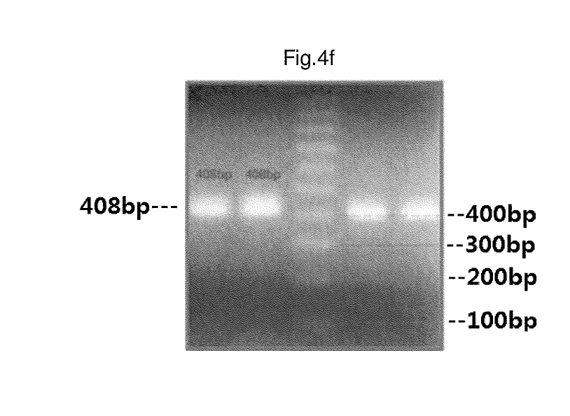

As shown in FIG. 3, T7 gene was divided into three parts and then was amplified by PCR respectively. FIGS. 4a to 4f show the PCR protocol of T7 bacteriophage gene. FIG. 4a-\textcircled{1} corresponds to 33.2 kb of a front region in 37.2 kb of T7 gene. FIG. 4a-\textcircled{2} corresponds to a middle region 400 bp in 37.2 kb T7 gene with the 6× His tag, which is purchased from Cosmogenetech (Seoul, Korea) in a form of plasmid. FIG. 4a-\textcircled{3} is a back region 3.6 kb in the T7 gene.

TABLE 1

| | | | SEQ ID |
|---|---|---|---|
| Designation | Template | Sequence 5′ → 3′ | NO: |
| forward primer 1 | Front region | TCTCACAGTGTACGGACCTAAAGT | 9 |
| | | TCCCCCATAG | |
| reverse primer 1 | Front region | TACCATCAGT GGCCACAACG | 10 |
| | | GCCTGACCTAC | |
| forward primer 2 | Middle region | TAGGTCAGGCCGTTGTGGCCACTG | 11 |
| | | ATG | |
| reverse primer 2 | Middle region | GAGAGTCCATCCGCGGACTACAC | 12 |
| | | GTC | |
| forward primer 3 | Back region | GACGTGTAGTCCGCGGATGGACT | 13 |
| | | CTC | |
| reverse primer 3 | Back region | AGGGACACAGAGAGACACTCAAG | 14 |
| | | GTAACACC | |

To carry out PCR, 20 pmol of the primers (Forward Primer 1 and Backward Primer 1), T7 gene 10 ng, 25 mM of dNTP, 5 μl of PCR buffer and 0.4 μl of PCR enzyme were mixed in PCR tube. Then, PCR was performed according to the temperature cycle below.

TABLE 2

| Segment | Cycle | Temperature | Time | |
|---|---|---|---|---|
| 1 | 1 | 93° C. | 3 | min |
| 2 | 10 | 93° C. | 15 | sec |
| | | Tm-5° C. | 30 | sec |
| | | 68° C. | 1 | min/kb |
| 3 | 25 | 93° C. | 15 | sec |
| | | Tm-5° C. | 30 | sec |

| | | | |
|---|---|---|---|
| | | 68° C. | 1 min/kb (+20 sec/cycle) |
| 4 | 1 | 4° C. | ∞ |

For each PCR amplified product, the PCR amplification was confirmed by performing agarose gel electrophoresis. The amplification of template gene was confirmed by identifying the presence of dark band.

1-2: Ligation of FIG. 4a-\textcircled{2} 400 by and FIG. 4a-\textcircled{3} 3.6 kb

Next, T7 gene including 6× His tag was made by ligating the three amplified genes. 400 by of FIG. 4a-\textcircled{2} and 3.6 kb of FIG. 4a-\textcircled{3} were connected by using PCR ligation method, and new second PCR product of 4 kb of FIG. 4a-\textcircled{4} and the product of FIG. 4-\textcircled{1} were connected by using the ligation kit purchased from Merck (Merck KGaA, Darmstadt, Germany), to obtain T7 gene including 6× His tag.

Two genes of the ligated FIG. 4a-\textcircled{2} 400 by and FIG. 4-\textcircled{3} 3.6 kb were ligated by PCR ligation method. Primers for PCR ligation of a forward primer 3 and a reverse primer 3 shown in Table 1 were used.

1 ul of 400 bp PCR product of FIG. 4a-\textcircled{2}, 1 ul PCR product of FIG. 4a-\textcircled{3} 3.6 kb, dNTP 25 mM, 5 ul of PCR buffer and 0.4 ul of PCR enzyme were mixed in PCR tube. Then PCR was performed on temperature cycle as the PCR experiment.

1-3: Ligation of FIG. 4a-\textcircled{4} 4 kb and FIG. 4a-\textcircled{1} 33.2 kb

Each ends of the second PCR product of FIG. 4a-\textcircled{4} 4 kb and FIG. 4a-\textcircled{1} 33.2 kb was cleaved with Sfi I of the single restriction enzyme. Two genes with the sticky ends were ligated with a ligation enzyme to produce 37.2 kg of T7 gene including 6× His tag.

The oligonucleotide and the templates of T7 bacteriophage were purified by performing electrophoresis to remove other impurities such as salts or enzyme etc, with a purification kit.

T4 DNA ligase 1 ul, 10 mM, ATP 0.5 ul, and DTT 0.5 ul were put into a tube containing oligonucleotide 0.05 pmol and T7 bacteriophage template 0.02 mol, and then were reacted by incubating at a 16° C. for four hours.

### Example 2

### Preparation of Modified T7 Bacteriophage Vector

2-1: T7 Bacteriophage Vector Connected Protein G

Protein G gene purchased from Promega was introduced by restriction site of EcoR I at N-terminal and Restriction site of Hind III at C-terminus. For performing the above experiment, PCR of protein G gene as a template was performed by designing two primers as follows. Forward primer 4 included restriction site of EcoR I and Reverse primer 4 included Restriction site of Hind III.

Forward primer 4(SEQ ID NO 15):
5′-GCTGAATTCATGACTTACAAA-3′
Reverse primer 4(SEQ ID NO 16):
5′-AAGCTTTTAT TCAGTTACCG-3′

After PCR, the protein G gene having restriction sites was purified by performing 1% agarose gel electrophoresis with a purification kit manufactured by Qiagen.

T7 bacteriophage vector where 6× His Tag was connected to tail gene was cleaved with two restriction enzymes of EcoR I and Hind III. After cleaving, the product was separated by electrophoresis in 0.5% agarose gel and then the band was purified.

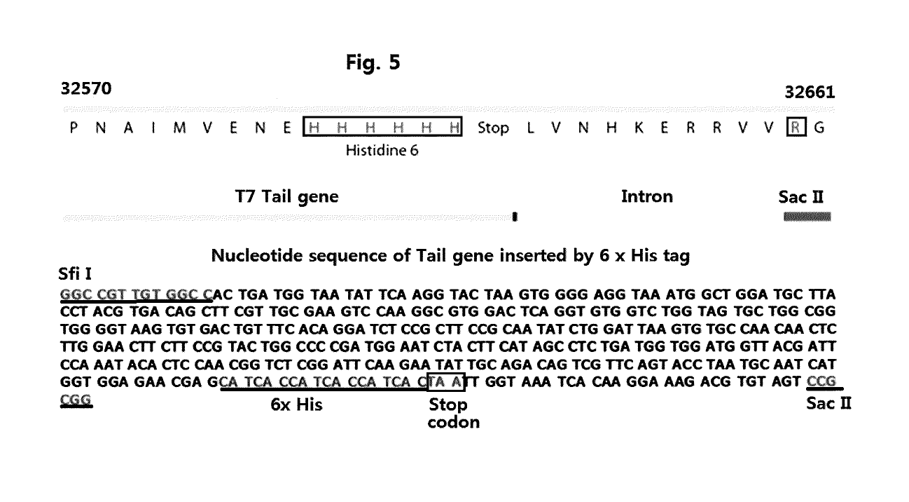

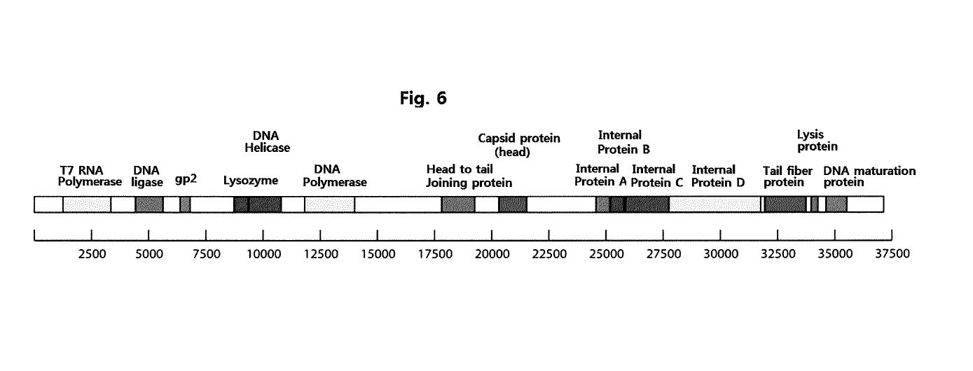

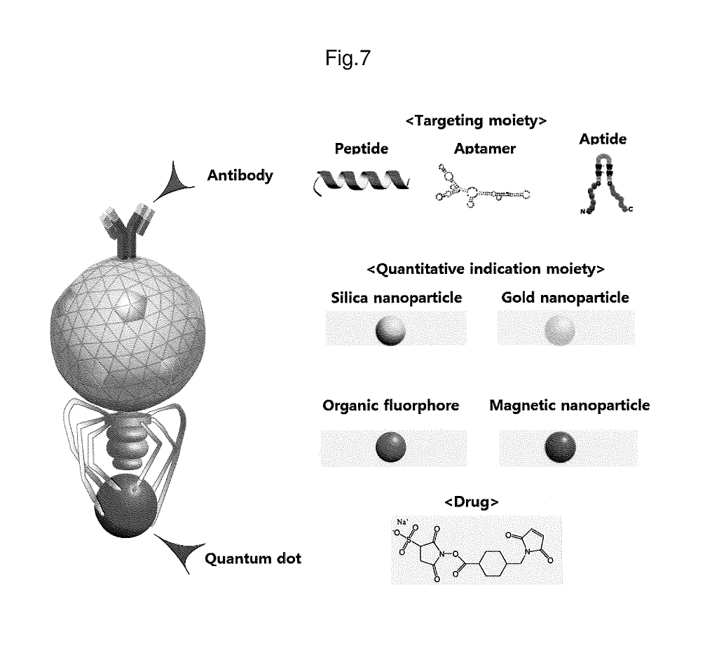

For genetic binding of T7 bacteriophage vector, the phosphate group at 5′-terminal was eliminated by using alkaline phophatase. The nucleotide sequence including the nucleotide sequence coding six HIS on tail part of T7 bacteriophage was shown in FIG. 5 and SEQ ID NO: 6.

2-2: Modified T7 Bacteriophage Vector

Protein G and T7 bacteriophage were ligated by adding 0.05 pmol of Protein G having restriction site and 0.02 pmol of T7 bacteriophage vector into a mixture of 1 μl of T4 DNA ligase (Novagen Inc. (Germany) 69839), 10 mM, ATP 0.5 μl, and DTT 0.5 μl (see the cleavage map of vector in FIG. 6).

### Example 3

### Producing and Culturing of Modified T7 Bacteriophage

3-1: Cell Transformation

BLT5403 competent cells were melted on ice, and 40 μl of cells were mixed with 2 μl of protein G-T7 bacteriophage-(HIS)tag in Example 2-3 in 1.5 ml polypropylene tube. After reacting on ice for 1 minute, the product was transferred to 0.1 cm Electroporation cuvette and given once with an electric shock by using Micropulser™ Electroporation Apparatus (Bio-rad, USA) under the condition of 1.8 kV, 4 ms, 200 Ohm and 25 μF.

After electrophoresis, the mixture was added by 1 ml of M9LB medium and incubated at 37° C. for 1 hour, to restore the cells.

3-2: Identifying the Production of T7 Bacteriophage

To check the production of bacteriophage in the electroporation, the plaque assay was carried out. 300 μl of the sample treated with electroporation was mixed with 3 ml of top agarose at 45 to 50° C., smeared on the plate including the ampicillin-added LB medium, and incubated at 37° C. for 3 to 4 hours. The production of bacteriophage was checked by counting the number of transparent plaque.

To further check the production of T7 bacteriophage, the liquid lysate amplification was performed. Specifically, one colony of BLT5403 E. coli was mixed with 50 mL of LB solution added with ampicillin, and was incubated at 200 rpm, 37° C. in incubator until O.D. (Optical Density) 600 reached 0.5 to 1.0. After mixing culture medium and 300 uL of sample made after electroporation, it was incubated at 200 rpm at 37° C. for 1 to 3 hours in incubator until the mixed solution was become transparent and O.D. value decreased. E. coli and bacteriophage were separated by centrifuging the obtained culture medium at 8,000 g for 10 minutes, and the supernatant was transferred to sterilized bottle and stocked at 4° C.

3-3: Selective Separation of T7 Bacteriophage with 6× His Tag

T7 bacteriophage with 6× His Tag on tail part was selectively separated by using the affinity selection method.

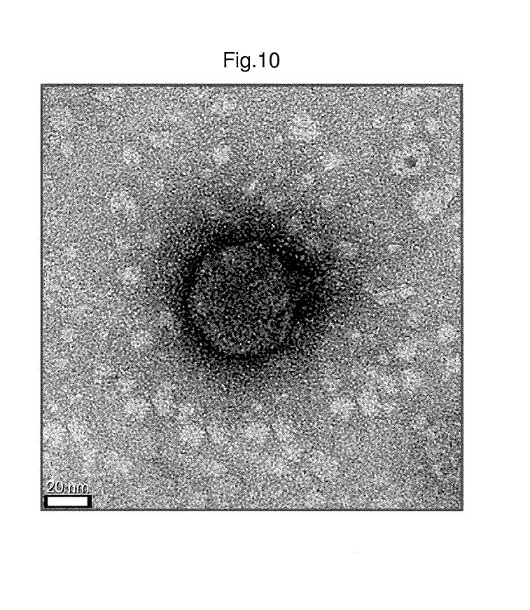

In particular, a piece of Ni madreporite (mesh=800 nm) was put in 50 ml of T7 lysate solution and reacted at 4° C. for 1 hour. The Ni madreporite was poured into polypropylene column which was coated by epoxy resin having a hole of 0.5 um diameter, and was washed by flowing 10 ml of 70% ethanol. The solution was collected by cleaving the bond formed between Histidine of T7 bacteriophage and Ni madreporite with 3 ml of 1 M of imidazole solution. The solution buffer was replaced with PBS by using amicon ultra purification filter (UFC510024, Millipore (USA)). T7 bacteriophage having 6× His tag was only separated selectively. TEM image of genetically-modified T7 bacteriophage was shown in FIG. 10.

### Example 4

### Preparation of Target-Specific Probe

4-1: Target-Specific Probe

1 pmol of recombinant T7 bacteriophage solution obtained in Example 3 was mixed with 1 pmol of anti-claudin4 rabbit monoclonal antibody solution, and was incubated at 4° C. for 1 hour, to obtain the target-specific probe with the targeting antibody.

4-2: Preparation of Labeling Agent

1 pmol of hydrophilic quantum dot coated with a mixture of 5% Ni-NTA lipid and 15% PEG2000 lipid, and 80% MHPC(1-myristoyl-2-hydroxy-sn-glycero-phosphocholine) lipid was put in the mixed solution of Example 4-1, and was reacted at 4° C. for one day for coupling the Histidine of T7 with Ni-NTA lipid part of the hydrophilic quantum dot.

4-3: Cell Preparation

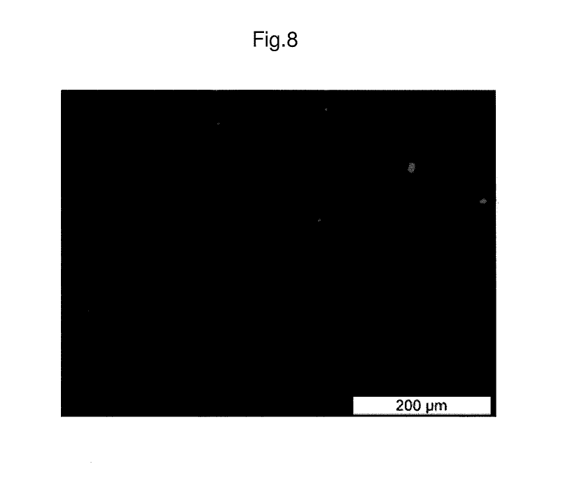

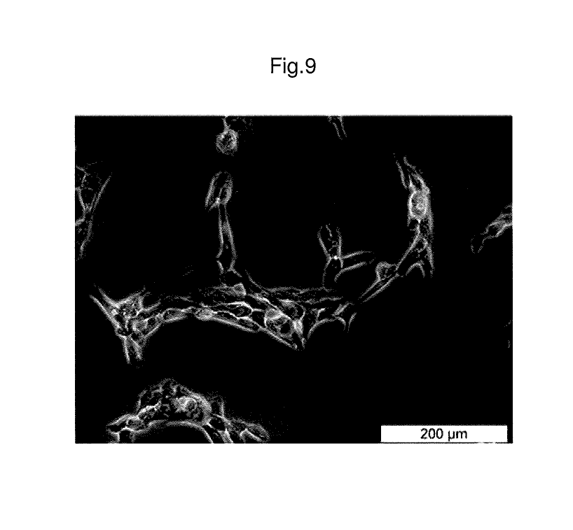

For preparing cells to be used for imaging, Capan-1 cells having a distribution of 4000 cells/cm² in T-25 flask were incubated under the condition of 37° C., 5% CO₂ for three days, and were fixed with 3.7% formaldehyde. The prepared cells were added by T7 bacteriophage having quantum dot and targeting antibody in 5% serum and 50 mM of NH₄Cl buffer, reacted at 37° C., 5% CO₂ for 8 hours, and washed by PBS solution at three times. The image was observed in the condition of TRITC filter, 100 ms exposing time with Leica (Leica, DP72) fluorescence microscope. FIG. 8 and FIG. 9 are fluorescence images of the cells targeted by using the genetically-modified T7 bacteriophage.

### Example 5

### Detection of Biomarker

1 mg/ml of capture antibody(anti-claudin4 rabbit monoclonal antibody) was put into 96-well plate, incubated at 25° C. for 2 hours and washed with 200 μl of PBS buffer at three times. The resulting solution was added by 300 μl of skim milk, and was reacted at 25° C. for 1 hour to prevent the nonspecific reaction. After removing the skimmed milk, 100 μl of serum containing antigen was added into the well. 100 μl of functionalized T7 bacteriophage with antibody and quantum dot was added to the well and incubated at 25° C. for 1 hour, and was washed with PBS.

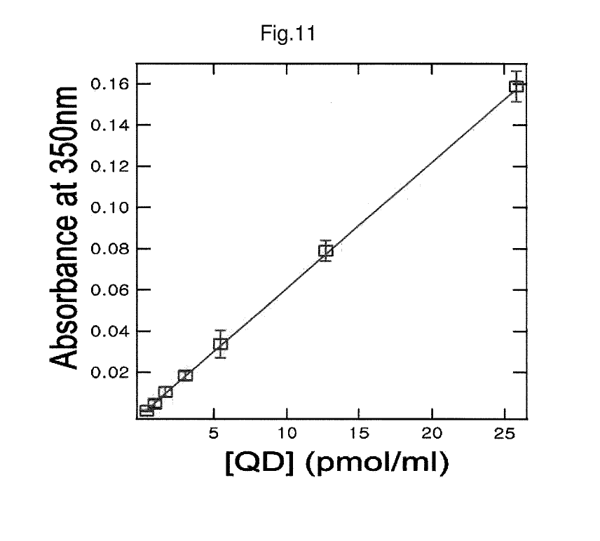

A standard curve was made by measuring an absorbance value at 350 nm wavelength of quantum dot of which the concentration was already determined (0.37 nM, 0.9 nM, 1.6 nM, 3 nM, 5 nM, 12.5 nM, 25 nM). The standard curve was shown in FIG. 11, depending on the concentration of quantum dot.

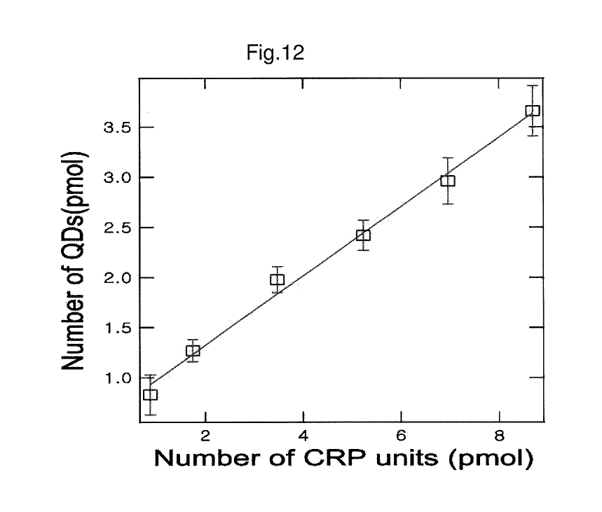

Then, the quantum dot coupled with T7 bacteriophage was separated by adding 100 μl of 1M imidazole to the sandwich assay and was incubated at room temperature for 30 minutes. The separated quantum dot of which the number corresponded to the number of antigen molecule was obtained by collecting supernatant, and was analyzed for the quantitative analysis by measuring absorbance of the solution and comparing the standard curve. The result of quantitative analysis of CRP biomarker was shown in FIG. 12.

As shown in FIG. 12, the result of quantitative analysis confirmed that the amount of detected quantum dot was proportional to the amount of CRP protein used in experiment. Thus, it was possible not only to detect the proteins at pico(10⁻¹²) molar level, but also to quantify the proteins accurately with T7 probe of the present invention.

What is claimed is:

1. A target-specific probe including a binding peptide bound to a head part of T7 bacteriophage, a targeting antibody connected to the binding peptide, and a detectable labeling agent bound to a tail part of T7 bacteriophage.

2. The target-specific probe according to claim 1, wherein the binding peptide is protein G, protein A, protein A/G, Fc receptor, protein Z or a biotinylation tag.

3. The target-specific probe according to claim 1, wherein the targeting antibody is connected to the binding peptide through Fc part of the targeting antibody.

4. The target-specific probe according to claim 1, wherein the binding peptide is provided by a fusion peptide where the binding peptide is bound to a C-terminus of the head part of T7 bacteriophage.

5. The target-specific probe according to claim 1, wherein the targeting antibody is bound to the binding peptide via HIS Tag, CYS Tag, GST Tag or biotinylation binding tag.

6. The target-specific probe according to claim 1, wherein the labeling agent is bound to the tail part of T7 bacteriophage via HIS Tag, CYS Tag, GST Tag or biotinylation binding tag.

7. The target-specific probe according to claim 6, wherein the tag is inserted into the internal site in the tail part of T7 bacteriophage or connected to an end of the tail part of T7 bacteriophage.

8. The target-specific probe according to claim 1, wherein the T7 bacteriophage is an modified T7 bacteriophage including a modified tail part with HIS Tag, CYS Tag, GST Tag or biotinylation tag, and a modified head part with the binding peptide connected to the C-terminus of the head part in T7 bacteriophage.

9. The target-specific probe according to claim 1, wherein the labeling agent is bound to the T7 bacteriophage at a molar ratio of 1:1.

10. The target-specific probe according to claim 1, wherein the labeling agent is a quantum dot, a magnetic bead nanoparticle, a gold nanoparticle, a fluorescent dye, a fluorescent protein, a nanophosphor, or a silicon nanoparticle.

11. The target-specific probe according to claim 10, wherein the quantum dot comprises a hydrophilic surface of amphiphilic material having a hydrophobic group and a hydrophilic group binding to the tail part of T7 bacteriophage.

12. The target-specific probe according to claim 11, wherein the amphiphilc material is at least one selected from the group consisting of MHPC, DPPE-PEG 2000, Ni-NTA and a mixture thereof.

13. A method for detecting a biomarker including:
contacting a target-specific probe including a targeting antibody being specific to the biomarker and a labeling agent obtained according to claim 7, with a sample containing a biomarker, and
detecting the labeling agent of the probe targeted to the biomarker.

14. The method for detecting a biomarker according to claim 13, further comprises determining the presence of biomarker in the sample, if the labeling agent is detected.

15. The method for detecting a biomarker according to claim 13, wherein the method further including a step of performing a quantitative analysis by comparing the amount of the detected labeling agent of the probe targeted to the biomarker, with a standard curve which is drawn with the amounts measured at various concentrations of the labeling agent.

16. The method for detecting a biomarker according to claim 13, wherein the fluorescence intensity emitted by quantum dot as the labeling agent is detected in the detection step.

17. A detection kit for a biomarker including a target-specific probe including a targeting agent and a labeling agent according to claim 7; and a detector detecting the labeling agent of the probe targeted to the biomarker.


SEQUENCE LISTING (16 sequences)

SEQ ID NO: 1 - protein, 195 aa, Artificial Sequence
  Binding peptide derived from ProG
TYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTE

SEQ ID NO: 2 - protein, 508 aa, Artificial Sequence
  Binding peptide derived from ProA
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNKFNKDQQSAFYEILNMPNLNEEQRNGFIQSLKDDPSQSTNVLGEAKKLNESQAPKADNNFNKEQQNAFYEILNMPNLNEEQRNGFIQSLKDDPSQSANLLAEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDGNKPGKEDGNKPGKEDNKKPGKEDGNKPGKEDNKKPGKEDGNKPGKEDGNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 3 - protein, 13 aa, Artificial Sequence
  BIOTINYLATING tag
LAAIPGAGLIGTH

SEQ ID NO: 4 - DNA, 1008 bp, Bacteriophage T7
  Head part of wild type T7 bacteriophage
atggctagcatgactggtggacagcaaatgggtactaaccaaggtaaaggtgtagttgctgctggagataaactggcgttgttcttgaaggtatttggcggtgaagtcctgactgcgttcgctcgtacctccgtgaccacttctcgccacatggtacgttccatctccagcggtaaatccgctcagttccctgttctgggtcgcactcaggcagcgtatctggctccgggcgagaacctcgacgataaacgtaaggacatcaaacacaccgagaaggtaatcaccattgacggtctcctgacggctgacgttctgatttatgatattgaggacgcgatgaaccactacgacgttcgctctgagtatacctctcagttgggtgaatctctggcgatggctgcggatggtgcggttctggctgagattgccggtctgtgtaacgtggaaagcaaatataatgagaacatcgagggcttaggtactgctaccgtaattgagaccactcagaacaaggccgcacttaccgaccaagttgcgctgggtaaggagattattgcggctctgactaaggctcgtgcggctctgaccaagaactatgttccggctgctgaccgtgtgttctactgtgacccagatagctactctgcgattctggcagcactgatgccgaacgcagcaaactacgctgctctgattgaccctgagaagggttctatccgcaacgttatgggctttgaggttgtagaagttccgcacctcaccgctggtggtgctggtaccgctcgtgagggcactactggtcagaagcacgtcttccctgccaataaaggtgagggtaatgtcaaggttgctaaggacaacgttatcggcctgttcatgcaccgctctgcggtaggtactgttaagctgcgtgacttggctctggagcgcgctcgccgtgctaacttccaagcggaccagattatcgctaagtacgcaatgggccacggtggtcttcgcccagaagct

SEQ ID NO: 5 - DNA, 1662 bp, Bacteriophage T7
  Tail part of wild type T7 bacteriophage
atggctaacgtaattaaaaccgttttgacttaccagttagatggctccaatcgtgattttaatatcccgtttgagtatctagcccgtaagttcgtagtggtaactcttattggtgtagaccgaaaggtccttacgattaatacagactatcgctttgctacacgtactactatctctctgacaaaggcttggggtccagccgatggctacacgaccatcgagttacgtcgagtaacctccactaccgaccgattggttgactttacggatggttcaatcctccgcgcgtatgaccttaacgtcgctcagattcaaacgatgcacgtagcggaagaggcccgtgacctcactacggatactatcggtgtcaataacgatggtcacttggatgctcgtggtcgtcgaattgtgaacctagcgaacgccgtggatgaccgcgatgctgttccgtttggtcaactaaagaccatgaaccagaactcatggcaagcacgtaatgaagccttacagttccgtaatgaggctgagactttcagaaaccaagcggagggctttaagaacgagtccagtaccaacgctacgaacacaaagcagtggcgcgatgagaccaagggtttccgagacgaagccaagcggttcaagaatacggctggtcaatacgctacatctgctgggaactctgcttccgctgcgcatcaatctgaggtaaacgctgagaactctgccacagcatccgctaactctgctcatttggcagaacagcaagcagaccgtgcggaacgtgaggcagacaagctggaaaattacaatggattggctggtgcaattgataaggtagatggaaccaatgtgtactggaaaggaaatattcacgctaacgggcgcctttacatgaccacaaacggttttgactgtggccagtatcaacagttctttggtggtgtcactaatcgttactctgtcatggagtggggagatgagaacggatggctgatgtatgttcaacgtagagagtggacaacagcgataggcggtaacatccagttagtagtaaacggacagatcatcacccaaggtggagccatgaccggtcagctaaaattgcagaatgggcatgttcttcaattagagtccgcatccgacaaggcgcactatattctatctaaagatggtaacaggaataactggtacattggtagagggtcagataacaacaatgactgtaccttccactcctatgtacatggtacgaccttaacactcaagcaggactatgcagtagttaacaaacacttccacgtaggtcaggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagtaa

SEQ ID NO: 6 - DNA, 408 bp, Artificial Sequence
  Tailgene of bacteriophage T7 with insertion of 6X His tag
ggccgttgtggccactgatggtaatattcaaggtactaagtggggaggtaaatggctggatgcttacctacgtgacagcttcgttgcgaagtccaaggcgtggactcaggtgtggtctggtagtgctggcggtggggtaagtgtgactgtttcacaggatctccgcttccgcaatatctggattaagtgtgccaacaactcttggaacttcttccgtactggccccgatggaatctacttcatagcctctgatggtggatggttacgattccaaatacactccaacggtctcggattcaagaatattgcagacagtcgttcagtacctaatgcaatcatggtggagaacgagcatcaccatcaccatcactaattggtaaatcacaaggaaagacgtgtagtccgcgg

SEQ ID NO: 7 - protein, 404 aa, Mus musculus
  Mus musculus (Mouse) Fc receptor I
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVKIHRLQREKKYNLEVPLVSEQGKKANSFQQVRSDGVYEEVTATASQTTPKEAPDGPRSSVGDCGPEQPEPLPPSDSTGAQTSQS

SEQ ID NO: 8 - protein, 374 aa, Homo sapiens
  Homo sapiens (Human) Fc receptor I
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 9 - DNA, 34 bp, Artificial Sequence
  Forward primer of the front part of T7 bacteriophage
tctcacagtgtacggacctaaagttcccccatag

SEQ ID NO: 10 - DNA, 31 bp, Artificial Sequence
  Reverse primer of the front part of T7 bacteriophage
taccatcagtggccacaacggcctgacctac

SEQ ID NO: 11 - DNA, 27 bp, Artificial Sequence
  Forward primer of the center part of T7 bacteriophage
taggtcaggccgttgtggccactgatg

SEQ ID NO: 12 - DNA, 26 bp, Artificial Sequence
  Reverse primer of the center part of T7 bacteriophage
gagagtccatccgcggactacacgtc

SEQ ID NO: 13 - DNA, 26 bp, Artificial Sequence
  Forward primer of the end part of T7 bacteriophage
gacgtgtagtccgcggatggactctc

SEQ ID NO: 14 - DNA, 31 bp, Artificial Sequence
  Reverse primer of the end part of T7 bacteriophage
agggacacagagagacactcaaggtaacacc

SEQ ID NO: 15 - DNA, 21 bp, Artificial Sequence
  Forward primer of proteinG gene
gctgaattcatgacttacaaa

SEQ ID NO: 16 - DNA, 20 bp, Artificial Sequence
  Reverse primer of protein G gene
aagcttttattcagttaccg